(19) United States
(12) Patent Application Publication
(10) Pub. No.: US 2010/0240544 A1
(43) Pub. Date: Sep. 23, 2010

(54) Aptamer biochip for multiplexed detection of biomolecules

(76) Inventors: David J. Liu, Fremont, CA (US); Xing Su, Cupertino, CA (US)

Correspondence Address: Pillsbury Winthrop Shaw Pittman LLP;(INTEL), P.O. Box 10500, McLean, VA, 22102, US

(21) Appl. No.: 11/529,554

(22) Filed: Sep. 29, 2006

(51) Int. Cl. C40B 30/04; C07H 21/04; C12P 19/34; C40B 40/08

(52) U.S. Cl. 506/9; 536/24.3; 536/24.1; 435/91.2; 506/17

(57) ABSTRACT

The embodiments of the invention relate to an in situ generated and self-addressed aptamer biochip for the multiplexed detection of biomolecules. The inventive aptamer biochip uses sets of complementary probes to permit in situ generation and immobilization of aptamers on the aptamer biochip surface to form an addressable aptamer array. These aptamer biochip arrays can be used for detecting multiple biomolecules, especially those for disease signature pattern analysis.

### FIELD OF INVENTION

The embodiments of the invention relate to an in situ generated and self-addressed aptamer biochip for the multiplexed detection of biomolecules. The inventive aptamer biochip uses sets of complementary probes to permit in situ generation of addressable aptamer arrays (i.e., biochips). These arrays can be used for detecting multiple biomolecules, especially those for disease signature pattern analysis.

### BACKGROUND

Aptamers are widely regarded a new generation of affinity agents with functional characteristics that are similar to antibodies. For example, aptamers can bind to their cognate targets with nanomolar or picomolar binding affinity, matching or even superseding the affinities of antibodies. Due to these inherent properties, aptamers hold great promise in various biological and biomedical applications. For instance, aptamers can serve as designer matrices for affinity chromatography, biosensors, therapeutic agents, genetic switches, allosteric ribozymes, capture agents for microarray biochips in molecular detection, and so on.

The ability for aptamers to serve as capture agents on the surface of microarray biochips is pertinent to many potential applications associated with protein microarrays including antibody microarrays and antigen microarrays which can form the basis for aptamer biochips. Potential applications of the aptamer biochips include protein profiling (proteomics), drug screening (drug discovery), biomarker discovery and analysis (diagnostics), biosensing (food analysis, environmental analysis and biosafety), and basic research.

With the completion of the Human Genome Project and dawn of proteomics era, new tools are needed to analyze biological functions of proteins expressed in any given organism. Proteomics as a new scientific field faces many of challenges. For example, numerous biological transformations have been associated with proteins after translation affecting the functions of proteins. These include posttranslational modifications, such as phosphorylation, splicing, etc. In addition, the dynamic range of protein expression in a given organism is very broad. More sensitive detection and analysis technologies are needed to address the dynamic range problem. As profiling proteins in a given organism is quite challenging, a new class of aptamer biochips would alleviate this burden by providing more sensitive detection technology, high density array, and high volume of information.

Aptamers can be designed to bind with specific target proteins and inactivate their targets. This ability suggests that aptamers may be used as surrogate ligands in high throughput screening (HTS) for drug discovery. The structural and functional information stored in the aptamer can be converted into a small molecule drug that can inhibit the target protein function and validate the drug target. Therefore, an aptamer biochip that permits multiplexed detection of biomolecules and in situ generated and self addressed aptamers would be a valuable tool for drug discovery and target validation.

Biomarker discovery for diseases poses a challenge to diagnostics research and development. Aptamer biochips provide a tool to discover new markers for diseases such as cancers and heart diseases. Further, aptamer biochips may pattern analysis of protein markers involved in a disease stage, providing a better overall picture which could be applied to improved diagnoses. For that reason, biomarker signature analysis is a growing trend in diagnostics industry.

The high binding affinity of aptamers to their cognate targets can be used in biosensing as well. For example, aptamer beacons can be built onto chip. Upon binding to their targets, the binding events will trigger a fluorescent emission of a specific wavelength. This technique could be used in food safety and environmental analysis. It is also useful for remote biosensing in a bio-threatening environment.

Basic research using aptamers as detecting agents in affinity assay such as ELISA, microarray, and immunoprecipitation, can be conducted to identify previously unknown proteins on cell surfaces, or inside cells; and especially to identify new disease markers. They can also be used detect proteins with very low abundance.

The application of aptamer biochips is not only for protein-related research and development, but also for other biological molecules including nucleic acids and small molecules, and even cells.

### BRIEF DESCRIPTION OF THE DRAWINGS

FIG. 1 is a representative illustration of an embodiment of the aptamer biochip of the present invention and the process of producing an aptamer biochip of the present invention.

FIG. 2 is an exemplary illustration of an aptamer biochip and its use.

FIG. 3 shows a design of one feature set for probe array.

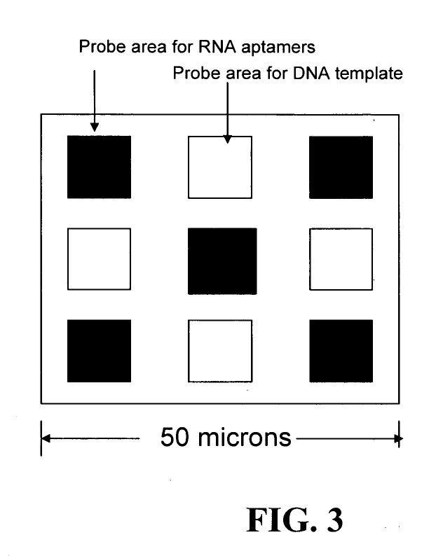

FIG. 4 shows in-situ transcribing of RNA aptamer molecule.

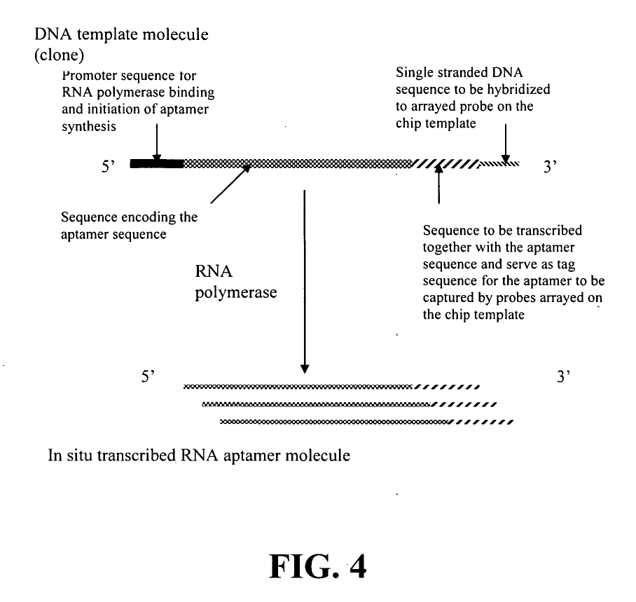

### DETAILED DESCRIPTION

As used in the specification and claims, the singular forms “a”, “an” and “the” include plural references unless the context clearly dictates otherwise. For example, the term “an array” may include a plurality of arrays unless the context clearly dictates otherwise. The following terms are used herein according to the definitions.

Nucleic acid means either DNA, RNA, single-stranded or double-stranded and any chemical modifications thereof, provided only that the modification does not interfere with amplification of selected nucleic acids. Such modifications include, but are not limited to, modifications at cytosine exocyclic amines, substitution of 5-bromo-uracil, backbone modifications, methylations, unusual base-pairing combinations and the like.

Ligand means a nucleic acid that binds another molecule (target). In a population of candidate nucleic acids, a ligand is one which binds with greater affinity than that of the bulk population. In a candidate mixture, more than one ligand could exist for a given target. The ligands can differ from one another in their binding affinities for the target molecule.

Candidate mixture is a mixture of nucleic acids of differing sequence, from which to select a desired ligand. The source of a candidate mixture can be from naturally-occurring nucleic acids or fragments thereof, chemically synthesized nucleic acids, enzymatically synthesized nucleic acids or nucleic acids made by a combination of the foregoing techniques.

Target molecule means any compound of interest for which a ligand is desired. A target molecule can be a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc., without limitation.

Partitioning means any process whereby ligands bound to target molecules, termed ligand-target pairs herein, can be separated from nucleic acids not bound to target molecules. Partitioning can be accomplished by various methods known in the art. Nucleic acid-protein pairs can be bound to nitrocellulose filters while unbound nucleic acids are not. Columns which specifically retain ligand-target pairs (or specifically retain bound ligand complexed to an attached target) can be used for partitioning. Liquid-liquid partition can also be used as well as filtration gel retardation, and density gradient centrifugation. The choice of partitioning method will depend on properties of the target and of the ligand-target pairs and can be made according to principles and properties known to those of ordinary skill in the art.

Amplifying means any process or combination of process steps that increases the amount or number of copies of a molecule or class of molecules. Amplifying RNA molecules may be carried out by a sequence of three reactions: making cDNA copies of selected RNAs, using polymerase chain reaction to increase the copy number of each cDNA, and transcribing the cDNA copies to obtain RNA molecules having the same sequences as the selected RNAs. Any reaction or combination of reactions known in the art can be used as appropriate, including direct DNA replication, direct RNA amplification and the like, as will be recognized by those skilled in the art. The amplification method should result in the proportions of the amplified mixture being essentially representative of the proportions of different sequences in the initial mixture.

Randomized is a term used to describe a segment of a nucleic acid having, in principle, any possible sequence over a given length. Randomized sequences may be of various lengths, as desired, ranging from about eight to more than 100 nucleotides (“nt”). The chemical or enzymatic reactions by which random sequence segments are made may not yield mathematically random sequences due unknown biases or nucleotide preferences that may exist. The term “randomized” is used instead of “random” to reflect the possibility of such deviations from non-ideality. In the techniques presently known, for example sequential chemical synthesis, large deviations are not known to occur. For short segments of 20 nucleotides or less, any minor bias that might exist would have negligible consequences. The longer the sequences of a single synthesis, the greater the effect of any bias.

A bias may be deliberately introduced into randomized sequence, for example, by altering the molar ratios of precursor nucleoside (or deoxynucleoside) triphosphates of the synthesis reaction. A deliberate bias may be desired, for example, to approximate the proportions of individual bases in a given organism, or to affect secondary structure.

SELEXION refers to a mathematical analysis and computer simulation used to demonstrate the powerful ability of SELEX to identify nucleic acid ligands and to predict which variations in the SELEX process have the greatest impact on the optimization of the process. SELEXION is an acronym for Systematic Evolution of Ligands by EXponential enrichment with Integrated Optimization by Nonlinear analysis.

A nucleic acid antibody is a term used to refer to a class of nucleic acid ligands that are comprised of discrete nucleic acid structures or motifs that selectively bind to target molecules. Nucleic acid antibodies may be made up of double or single stranded RNA or DNA. The nucleic acid antibodies are synthesized, and in a preferred embodiment are constructed based on a ligand solution or solutions received for a given target by the SELEX process. In many cases, the nucleic acid antibodies of the present invention are not naturally occurring in nature, while in other situations they may have significant similarity to a naturally occurring nucleic acid sequence.

The nucleic acid antibodies of the present invention include all nucleic acids having a specific binding affinity for a target, while not including the cases when the target is a polynucleotide which binds to the nucleic acid through a mechanism which predominantly depends on Watson/Crick base pairing or triple helix agents (See, Riordan, M. et al. (1991) Nature 350:442-443); provided, however, that when the nucleic acid antibody is double-stranded DNA, the target is not a naturally occurring protein whose physiological function depends on specific binding to double-stranded DNA.

An RNA motif is a term generally used to describe the secondary or tertiary structure of RNA molecules. The primary sequence of RNA is a specific string of nucleotides (A, C, G or U) in one dimension. The primary sequence does not give information on first impression as to the three dimensional configuration of the RNA, although it is the primary sequence that dictates the three dimensional configuration. In certain cases, the ligand solution obtained after performing SELEX on a given target may best be represented as a primary sequence. Although conformational information pertaining to such a ligand solution is not always ascertainable based on the results obtained by SELEX, the representation of a ligand solution as a primary sequence shall not be interpreted as disclaiming the existence of an integral tertiary structure.

As used herein, the term “aptamer” or “specifically binding oligonucleotide” refers to an oligonucleotide that is capable of forming a complex with an intended target substance. The complexation is target-specific in the sense that other materials which may accompany the target do not complex to the aptamer. It is recognized that complexation and affinity are a matter of degree; however, in this context, “target-specific” means that the aptamer binds to target with a much higher degree of affinity than it binds to contaminating materials.

Generally, aptamers are macromolecules composed of nucleic acid, such as RNA or DNA that bind tightly to a specific molecular target. As is typical of nucleic acids, a particular aptamer may be described by a linear sequence of nucleotides (A, U or T, C and G). These sequences are generally about 15-60 letters long. In practice, however, the chain of nucleotides forms intramolecular interactions that result in a molecule with a complex three-dimensional shape. The shape of the aptamer contributes to its ability to bind tightly against with surface of its target molecule. Since a tremendous range of molecular shapes exist among the possibilities for nucleotide sequences, aptamers may be obtained for a wide array of molecular targets, including most proteins and many small molecules.

The aptamer may be prepared by any known method, including synthetic, recombinant, and purification methods, and may be used alone or in combination with other aptamers specific for the same target. Further, as described more fully herein, the term “aptamer” specifically includes “secondary aptamers” containing a consensus sequence derived from comparing two or more known aptamers to a given target.

As used herein, the term “binding” refers to an interaction or complexation between a target and an oligonucleotide or aptamer, resulting in a sufficiently stable complex so as to permit separation of oligonucleotide/target complexes from uncomplexed oligonucleotides under given binding complexation or reaction conditions. Binding is mediated through hydrogen bonding or other molecular forces. As used herein, the term “binding” specifically excludes the normal “Watson-Crick”-type binding interactions (i.e., adenine-thymine and guanine-cytosine base-pairing) traditionally associated with the DNA double helix.

The term “specific binding” or “specific interaction” is the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme—substrate interactions, polynucleotide hybridization interactions, and so forth.

Specific binding is a term may also be defined on a case-by-case basis. In the context of a given interaction between a given ligand and a given target, a binding interaction of ligand and target of higher affinity than that measured between the target and the candidate ligand mixture is observed. In order to compare binding affinities, the conditions of both binding reactions should be the same, and should be comparable to the conditions of the intended use. For the most accurate comparisons, measurements can be made that reflect the interaction between ligand as a whole and target as a whole. Nucleic acid ligands can be selected to be as specific as required, either by establishing selection conditions that demand the requisite specificity during SELEX, or by tailoring and modifying the ligand through “walking” and other modifications using interactions of SELEX.

Aptamers, for example, can be used as a separation tool for retrieving the targets to which they specifically bind. In these methods, the aptamers function much like monoclonal antibodies in their specificity and usage. By coupling the aptamers containing the specifically binding sequences to a solid support, desired target substances can be recovered in useful quantities. In addition, these aptamers can be used in diagnosis by employing them in specific binding assays for the target substances. When suitably labeled using detectable moieties such as radioisotopes, the specifically binding oligonucleotides can also be used for in vivo imaging or histological analysis.

Furthermore, when the aptamer specifically binds to biologically active sites on a biomolecule, that aptamer can be used therapeutically to affect that biological activity.

“Oligomers” or “oligonucleotides” include RNA or DNA sequences of more than one nucleotide in either single chain or duplex form and specifically includes short sequences such as dimers and trimers, in either single chain or duplex form, which may be intermediates in the production of the specifically binding oligonucleotides. “Nucleic acids”, as used herein, refers to RNA or DNA sequences of any length in single-stranded or duplex form.

An “array,” “macroarray” or “microarray” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the sample spots on the array. A macroarray generally contains sample spot sizes of about 300 microns or larger and can be easily imaged by gel and blot scanners. A microarray could generally contain spot sizes of less than 300 microns.

“Solid support,” “support,” and “substrate” refer to a material or group of materials having a rigid or semi-rigid surface or surfaces. In some aspects, at least one surface of the solid support could be substantially flat, although in some aspects it may be desirable to physically separate synthesis regions for different molecules with, for example, wells, raised regions, pins, etched trenches, or the like. In certain aspects, the solid support(s) could take the form of beads, resins, gels, microspheres, or other geometric configurations.

A “nanomaterial” as used herein refers to a structure, a device or a system having a dimension at the atomic, molecular or macromolecular levels, in the length scale of approximately 1-100 nanometer range. Preferably, a nanomaterial has properties and functions because of the size and can be manipulated and controlled on the atomic level.

The term “target” or “target molecule” refers to a molecule of interest that is to be analyzed, e.g., a nucleotide, an oligonucleotide, or a protein. The target or target molecule could be a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to molecular probes such as chemically modified carbon nanotubes, carbon nanotube bundles, nanowires, nanoclusters or nanoparticles. The target molecule may be fluorescently labeled DNA or RNA.

The term “probe” or “probe molecule” refers to a molecule that binds to a target molecule for the analysis of the target. The probe or probe molecule is generally, but not necessarily, has a known molecular structure or sequence. The probe or probe molecule is generally, but not necessarily, attached to the substrate of the array. The probe or probe molecule is typically a nucleotide, an oligonucleotide, or a protein, including, for example, cDNA or pre-synthesized polynucleotide deposited on the array. Probes molecules are biomolecules capable of undergoing binding or molecular recognition events with target molecules. (In some references, the terms “target” and “probe” are defined opposite to the definitions provided here.) The polynucleotide probes require the sequence information of genes, and thereby can exploit the genome sequences of an organism. In cDNA arrays, there could be cross-hybridization due to sequence homologies among members of a gene family. Polynucleotide arrays can be specifically designed to differentiate between highly homologous members of a gene family as well as spliced forms of the same gene (exon-specific). Polynucleotide arrays of the embodiment of this invention could also be designed to allow detection of mutations and single nucleotide polymorphism. A probe or probe molecule can be a capture molecule.

The term “molecule” generally refers to a macromolecule or polymer as described herein. However, arrays comprising single molecules, as opposed to macromolecules or polymers, are also within the scope of the embodiments of the invention.

A “macromolecule” or “polymer” comprises two or more monomers covalently joined. The monomers may be joined one at a time or in strings of multiple monomers, ordinarily known as “oligomers.” Thus, for example, one monomer and a string of five monomers may be joined to form a macromolecule or polymer of six monomers. Similarly, a string of fifty monomers may be joined with a string of hundred monomers to form a macromolecule or polymer of one hundred and fifty monomers. The term polymer as used herein includes, for example, both linear and cyclic polymers of nucleic acids, polynucleotides, polynucleotides, polysaccharides, oligosaccharides, proteins, polypeptides, peptides, phospholipids and peptide nucleic acids (PNAs). The peptides include those peptides having either α-, β-, or ω-amino acids. In addition, polymers include heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which could be apparent upon review of this disclosure.

The term “nucleotide” includes deoxynucleotides and analogs thereof. These analogs are those molecules having some structural features in common with a naturally occurring nucleotide such that when incorporated into a polynucleotide sequence, they allow hybridization with a complementary polynucleotide in solution. Typically, these analogs are derived from naturally occurring nucleotides by replacing and/or modifying the base, the ribose or the phosphodiester moiety. The changes can be tailor-made to stabilize or destabilize hybrid formation, or to enhance the specificity of hybridization with a complementary polynucleotide sequence as desired, or to enhance stability of the polynucleotide.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide of the embodiments of the invention may be polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as “nucleotide polymers”.

An “oligonucleotide” may also be referred to as a polynucleotide having 2 to 20 nucleotides. Analogs also include protected and/or modified monomers as are conventionally used in polynucleotide synthesis. As one of skill in the art is well aware, polynucleotide synthesis uses a variety of base-protected nucleoside derivatives in which one or more of the nitrogens of the purine and pyrimidine moiety are protected by groups such as dimethoxytrityl, benzyl, tert-butyl, isobutyl and the like.

When the macromolecule of interest is a peptide, the amino acids can be any amino acids, including α, β, or ω-amino acids. When the amino acids are α-amino acids, either the L-optical isomer or the D-optical isomer may be used. Additionally, unnatural amino acids, for example, β-alanine, phenylglycine and homoarginine are also contemplated by the embodiments of the invention. These amino acids are well-known in the art.

A “peptide” is a polymer in which the monomers are amino acids and which are joined together through amide bonds and alternatively referred to as a polypeptide. In the context of this specification it should be appreciated that the amino acids may be the L-optical isomer or the D-optical isomer. Peptides are two or more amino acid monomers long, and often more than 20 amino acid monomers long.

A “protein” is a long polymer of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies.

The term “sequence” refers to the particular ordering of monomers within a macromolecule and it may be referred to herein as the sequence of the macromolecule.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” For example, hybridization refers to the formation of hybrids between a probe polynucleotide (e.g., a polynucleotide of the invention which may include substitutions, deletion, and/or additions) and a specific target polynucleotide (e.g., an analyte polynucleotide) wherein the probe preferentially hybridizes to the specific target polynucleotide and substantially does not hybridize to polynucleotides consisting of sequences which are not substantially complementary to the target polynucleotide. However, it could be recognized by those of skill that the minimum length of a polynucleotide desired for specific hybridization to a target polynucleotide could depend on several factors: G/C content, positioning of mismatched bases (if any), degree of uniqueness of the sequence as compared to the population of target polynucleotides, and chemical nature of the polynucleotide (e.g., methylphosphonate backbone, phosphorothiolate, etc.), among others.

Methods for conducting polynucleotide hybridization assays have been well developed in the art. Hybridization assay procedures and conditions could vary depending on the application and are selected in accordance with the general binding methods known in the art.

It is appreciated that the ability of two single stranded polynucleotides to hybridize could depend upon factors such as their degree of complementarity as well as the stringency of the hybridization reaction conditions.

As used herein, “stringency” refers to the conditions of a hybridization reaction that influence the degree to which polynucleotides hybridize. Stringent conditions can be selected that allow polynucleotide duplexes to be distinguished based on their degree of mismatch. High stringency is correlated with a lower probability for the formation of a duplex containing mismatched bases. Thus, the higher the stringency, the greater the probability that two single-stranded polynucleotides, capable of forming a mismatched duplex, could remain single-stranded. Conversely, at lower stringency, the probability of formation of a mismatched duplex is increased.

The appropriate stringency that could allow selection of a perfectly-matched duplex, compared to a duplex containing one or more mismatches (or that could allow selection of a particular mismatched duplex compared to a duplex with a higher degree of mismatch) is generally determined empirically. Means for adjusting the stringency of a hybridization reaction are well-known to those of skill in the art.

A “ligand” is a molecule that is recognized by a particular receptor. Examples of ligands that can be investigated by this invention include, but are not restricted to, agonists and antagonists for cell membrane receptors, toxins and venoms, viral epitopes, hormones, hormone receptors, peptides, enzymes, enzyme substrates, cofactors, drugs (e.g. opiates, steroids, etc.), lectins, sugars, polynucleotides, nucleic acids, oligosaccharides, proteins, and monoclonal antibodies.

A “receptor” is molecule that has an affinity for a given ligand. Receptors may-be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term “receptors” is used herein, no difference in meaning is intended. A “ligand receptor pair” is formed when two macromolecules have combined through molecular recognition to form a complex. Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value could be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmitters is useful in the development of drugs which can be used in the treatment of disorders of neurotransmission.

c) Antibodies: For instance, the invention may be useful in investigating the ligand-binding site on the antibody molecule which combines with the epitope of an antigen of interest; determining a sequence that mimics an antigenic epitope may lead to the development of vaccines of which the immunogen is based on one or more of such sequences or lead to the development of related diagnostic agents or compounds useful in therapeutic treatments such as for auto-immune diseases (e.g., by blocking the binding of the “anti-self” antibodies).

d) Nucleic Acids: Sequences of nucleic acids may be synthesized to establish DNA or RNA binding sequences.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant.

f) Hormone receptors: Examples of hormones receptors include, e.g., the receptors for insulin and growth hormone. Determination of the ligands which bind with high affinity to a receptor is useful in the development of, for example, an oral replacement of the daily injections which diabetics take to relieve the symptoms of diabetes. Other examples are the vasoconstrictive hormone receptors; determination of those ligands which bind to a receptor may lead to the development of drugs to control blood pressure.

g) Opiate receptors: Determination of ligands which bind to the opiate receptors in the brain is useful in the development of less-addictive replacements for morphine and related drugs.

The term “specific binding” or “specific interaction” is the specific recognition of one of two different molecules, for the other compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide hybridization interactions, and so forth.

The term “bi-functional linker group” refers to an organic chemical compound that has at least two chemical groups or moieties, such are, carboxyl group, amine group, thiol group, aldehyde group, epoxy group, that can be covalently modified specifically; the distance between these groups is equivalent to or greater than 5-carbon bonds.

The phrase “SERS active material,” “SERS active particle,” or “SERS cluster” refers to a material, a particle or a cluster of particles that produces a surface-enhanced Raman scattering effect. The SERS active material or particle generates surface enhanced Raman signal specific to the analyte molecules when the analyte-particle complexes are excited with a light source as compared to the Raman signal from the analyte alone in the absence of the SERS active material or SERS active particle. The enhanced Raman scattering effect provides a greatly enhanced Raman signal from Raman-active analyte molecules that have been adsorbed onto certain specially-prepared SERS active surfaces. The SERS active surface could be planar or curved. Typically, the SERS active surfaces are metal surfaces. Increases in the intensity of Raman signal could be in the order of 10⁴-10¹⁴ for some systems. SERS active material or particle includes a variety of metals including coinage (Au, Ag, Cu), alkalis (Li, Na, K), Al, Pd and Pt. In the case of SERS active particle, the particle size of SERS active particles could range from 1 to 5000 nanometers, preferably in the range of 5 to 250 nanometers, more preferably in the range of 10 to 150 nanometers, and most preferably 40 to 80 nanometers.

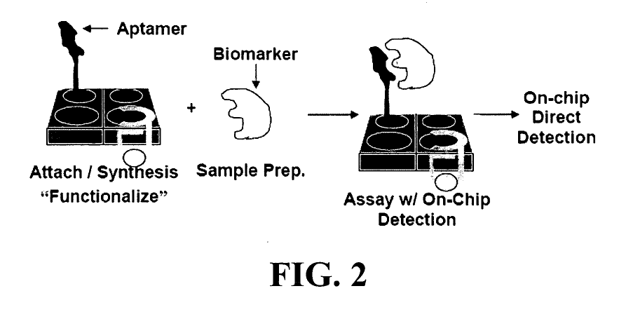

The term “capture particle” refers to a particle that can capture an analyte. The capture particle could be a coinage metal nanoparticle with surface modification to allow strong physical and/or chemical adsorption of analyte molecules and to allow adhesion of “enhancer particles” by electrostatic attraction, through specific interaction using a linker such as antibody-antigen, DNA hybridization, etc. or through the analyte molecule. An embodiment of a capture particle is shown in FIG. 2 wherein a metal particle has surface modification (shown as a hatched ring) and further has linkers that can combine with linkers on an enhancer particle.

The term “enhancer particle” refers to a SERS active particle with suitable surface modification, a linker or an analyte which combines with a capture particle to form an aggregate. In case the capture particle is positively charged, then a negatively charged SERS active particle can be used as an enhancer particle without a linker, and vise versa. In case the capture particle has an antigen or an antibody, then a SERS active particle having a complimentary linker, namely, an antibody or an antigen, could be used as an enhancer particle. An embodiment of an enhancer particle is shown in FIG. 2 wherein a metal particle has surface modification (shown as a hatched ring) and further has linkers that can combine with linkers on a capture particle.

The term “tagged particle” refers a SERS active particle having one or more different Raman active labels attached to the SERS active particle by direct attachment or through a surface modification. A tagged particle has a linker that can link to another tagged particle via an analyte. An embodiment of a tagged particle is shown in FIG. 2 wherein a metal particle has surface modification (shown as a hatched ring) and further has Raman active labels and linkers that can link to another tagged particle via an analyte.

The term “complementary” refers to the topological compatibility or matching together of interacting surfaces of a ligand molecule and its receptor. Thus, the receptor and its ligand can be described as complementary, and furthermore, the contact surface characteristics are complementary to each other.

The term “analyte” means any atom, chemical, molecule, compound, composition or aggregate of interest for detection and/or identification. Examples of analytes include, but are not limited to, an amino acid, peptide, polypeptide, protein, glycoprotein, lipoprotein, nucleoside, nucleotide, oligonucleotide, nucleic acid, sugar, carbohydrate, oligosaccharide, polysaccharide, fatty acid, lipid, hormone, metabolite, cytokine, chemokine, receptor, neurotransmitter, antigen, allergen, antibody, substrate, metabolite, cofactor, inhibitor, drug, pharmaceutical, nutrient, prion, toxin, poison, explosive, pesticide, chemical warfare agent, biohazardous agent, radioisotope, vitamin, heterocyclic aromatic compound, carcinogen, mutagen, narcotic, amphetamine, barbiturate, hallucinogen, waste product and/or contaminant. In certain embodiments of the invention, one or more analytes may be labeled with one or more Raman labels, as disclosed below. The sample such as an analyte in the embodiments of this invention could be in the form of solid, liquid or gas. The sample could be analyzed by the embodiments of the method and device of this invention when the sample is at room temperature and at lower than or higher than the room temperature.

The term “label” or “tag” is used to refer to any molecule, compound or composition that can be used to identify a sample such as an analyte to which the label is attached. In various embodiments of the invention, such attachment may be either covalent or non-covalent. In non-limiting examples, labels, may be fluorescent, phosphorescent, luminescent, electroluminescent, chemiluminescent or any bulky group or may exhibit Raman or other spectroscopic characteristics.

The terms “die,” “polymer array chip,” “DNA array,” “array chip,” “DNA array chip,” or “biochip” are used interchangeably and refer to a collection of a large number of probes arranged on a shared substrate which could be a portion of a silicon wafer, a nylon strip or a glass slide.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the sample spots on the array. A macroarray generally contains sample spot sizes of about 300 microns or larger and can be easily imaged by gel and blot scanners. A microarray would generally contain spot sizes of less than 300 microns.

“Predefined region”, “spot” “binding area” or “pad” refers to a localized area on a solid support which is, was, or is intended to be used for the formation of a selected molecule and is otherwise referred to herein in the alternative as a “selected” region. The predefined region may have any convenient shape, e.g., circular, rectangular, elliptical, wedge-shaped, etc. For the sake of brevity herein, “predefined regions” are sometimes referred to simply as “regions” or “spots.” In some embodiments, a predefined region and, therefore, the area upon which each distinct molecule is synthesized is smaller than about 1 cm² or less than 1 mm², and still more preferably less than 0.5 mm². In most preferred embodiments the regions have an area less than about 10,000 μm² or, more preferably, less than 100 μm². More preferably, a die of a wafer contains at least 400 spots in, for example, an at least 20×20 matrix. Even more preferably, the die contains at least 2048 spots in, for example, an at least 64×32 matrix, and still more preferably, the die contains at least 204,800 spots in, for example, an at least 640×320 array. A spot could contain an electrode to generate an electrochemical reagent, a working electrode to synthesize a polymer and a confinement electrode to confine the generated electrochemical reagent. The electrode to generate the electrochemical reagent could be of any shape, including, for example, circular, flat disk shaped and hemisphere shaped. In some aspects, a predefined region can be achieved by physically separating the regions (i.e., beads, resins, gels, etc.) into wells, trays, etc.

Microarray biochips can generally be described as consisting of small DNA fragments (referred to here as probes). The DNA fragments may be chemically synthesized at specific locations on a surface, such as coated quartz. A feature is the exact location where each probe is synthesized within the array. Within any given array, there may be millions of features.

Nucleic acids are typically extracted and labeled from experimental samples. The prepared samples are then hybridized to the array. The amount of label can be monitored at each feature, enabling a wide range of applications on a whole-genome scale—including gene- and exon-level expression analysis, novel transcript discovery, genotyping, and resequencing. Microarray analysis can also be combined with chromatin immunoprecipitation to perform genome-wide identification of transcription factors and their respective binding sites.

The manufacturing procedure of an aptamer biochip of the embodiments of the invention involves the following steps:

(1) Generating aptamer dsDNA clones or library with single stranded ends of unique sequences.

(2) Generating DNA probe chips.

(3) Hybridizing dsDNA to probes on the chip.

(4) Generating RNA aptamers by RNA polymerase (RNA transcription).

(5) Immobilizing RNA aptamers by self-addressed hybridization.

(6) Optionally removing dsDNA generating aptamer chips.

Therefore, there are two chips involved in this process. One is referred as “probe chip” (step 2 above) and the other as “aptamer chip.” The following discusses these two chips separately for clarity.

Probe Chip: This can be manufactured by photochemical or chemical process or by physical absorption. This chip can be manufactured by classic photolithography technologies developed in the prior art using a photomask and photoactive protecting groups. On this chip, preferably, each array spot contains two kinds of probes (primers), one for incoming aptamer dsDNA templates and the other one for RNA aptamers to be in situ synthesized. The length requirement of these probes is not very stringent and can be less than 25 nt. Alternatively, if the probes are pre-synthesized, they can be attached to an array surface using technologies of (a) micro-channel pumping; (b) “ink-jet” spotting; (c) template-stamping; and (d) photocrosslinking.

Aptamer Chip: This is manufactured by enzymatic process in situ. This is the core technology unique to this invention since the aptamers are generated on chip in situ through enzymatic synthesis, followed by self-addressed immobilization. Some of the features includes: (a) the aptamer length can be more than 100 nt. On contrary, traditional photolithography can normally produce 25 nt oligonucleotides on chip; (b) high density of aptamers on chip; and (c) high volume of information.

After probe chip is made, aptamer dsDNA clones that are selected with promoters and single stranded ends of unique sequences are hybridized to one set of the probes on probe chip. RNA polymerase, primers, and nucleoside triphosphates (NTPs) in proper buffer solution are then applied to the chip, initiating RNA transcription and generating RNA aptamers according to the instruction of aptamer dsDNA clones. The in situ generated RNA aptamers will be immobilized by self-addressed hybridization to another set of adjacent probes. The end product is an aptamer chip. Optionally, the dsDNA can be removed by applying DNase, leaving only RNA aptamers on chip.

RNA synthesis is at the hub of human genetic control. It could be used for understanding cancers, viral infections and normal human development. RNA is made from a DNA template. DNA is the genetic material—the blueprint for life. RNA is a transcript of the genetic coding in DNA. RNA transcripts are translated into proteins, which give a cell its identity. Programming of RNA synthesis determines cell fate, and mis-programming of RNA synthesis can lead to cancer or can support the life cycle of an invading virus.

RNA synthesis is similar to an industrial assembly line. RNA is assembled on a DNA template within an enzyme named RNA polymerase, a “nanomachine” for synthesis of RNA. RNA polymerase is named for its ability to “polymerize” an RNA chain from a DNA template. RNA polymerase works like a miniature RNA factory, reading the DNA code to assemble an RNA polymer from building blocks called “bases”, or in chemical terms “nucleoside triphosphates” (NTPs: ATP, GTP, CTP, UTP or A, G, C, and U). In the industrial assembly line model, DNA flows through the RNA polymerase like the conveyer belt in a factory. NTPs assemble with complementary bases on the DNA (A, G, C, and T). At the factory center, an RNA “polymer” is synthesized by continual addition of individual NTPs to a growing RNA chain. NTP substrates preload to the DNA template several steps before they are added to the growing RNA chain.

Preloading of NTP substrates maintains the accuracy of RNA synthesis. This is because the identity of incoming NTP substrates is checked and re-checked against the complementary DNA template before addition to the RNA chain. In this way, mistakes in transcribing DNA are minimized, and RNA is a highly accurate copy of the genetic information in DNA. For RNA synthesis, multiple fidelity checks are made before an RNA product is assembled.

In a preferred embodiment, nucleic acid ligands are derived from the SELEX process methodology. The SELEX process is described in U.S. Pat. No. 5,475,096 entitled “Nucleic Acid Ligands,” and in U.S. Pat. No. 5,270,163 (see also WO91/19813) entitled “Nucleic Acid Ligands,” which are incorporated herein by reference. The SELEX process provides a class of products which are nucleic acid molecules, each having a unique sequence, and each of which has the property of binding specifically to a desired target compound or molecule. Target molecules are preferably proteins (as in this application), but can also include among others carbohydrates, peptidoglycans and a variety of small molecules. SELEX methodology can also be used to target biological structures, such as cell surfaces or viruses, through specific interaction with a molecule that is an integral part of that biological structure.

In its most basic form, the SELEX process may be defined by the following series of steps:

1) A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are chosen either: (a) to assist in the amplification steps described below, (b) to mimic a sequence known to bind to the target, or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

2) The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.

3) The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 5-50%) are retained during partitioning.

4) Those nucleic acids selected during partitioning as having the relatively higher affinity for the target are then amplified to create a new candidate mixture that is enriched in nucleic acids having a relatively higher affinity for the target.

5) By repeating the partitioning and amplifying steps above, the newly formed candidate mixture contains fewer and fewer unique sequences, and the average degree of affinity of the nucleic acids to the target will generally increase. Taken to its extreme, the SELEX process will yield a candidate mixture containing one or a small number of unique nucleic acids representing those nucleic acids from the original candidate mixture having the highest affinity to the target molecule.

Many modifications of the basic SELEX process are known in the art. Such modifications may be made post-SELEX process (modification of previously identified unmodified ligands) or by incorporation into the SELEX process.

Nucleic acid ligands identified according to these methods have great utility in the field of biomedicine, including, but not limited to, use as diagnostic and prognostic reagents, as reagents for the discovery of novel therapeutics, as reagents for monitoring drug response in individuals, and as reagents for the discovery of novel therapeutic targets. It is contemplated that these methods can provide nucleic acid ligands that can be used in a microarray format, as set forth in the present biochip applications.

Biochip microarray production employs various semiconductor fabrication techniques, such as solid phase chemistry, combinatorial chemistry, molecular biology, and robotics. One process typically used is a photolithographic manufacturing process for producing microarrays with millions of probes on a single chip. Alternatively, if the probes pre-synthesized, they can be attached to an array surface using techniques such as micro-channel pumping, “ink-jet” spotting, template-stamping, or photocrosslinking.

An exemplary photolithographic process begins by coating a quartz wafer with a light-sensitive chemical compound to prevent coupling between the quart wafer and the first nucleotide of the DNA probe being created. A lithographic mask is used to either inhibit or permit the transmission of light onto specific locations of the wafer surface. The surface is then contacted with a solution which may contain adenine, thymine, cytosine, or guanine, and coupling occurs only in those regions on the glass that have been deprotected through illumination.

The coupled nucleotide bears a light-sensitive protecting group, allowing the cycle can be repeated. In this manner, the microarray is created as the probes are synthesized via repeated cycles of deprotection and coupling. The process may be repeated until the probes reach their full length, usually about 25 nucleotides. Commercially available arrays are typically manufactured at a density of over 1.3 million unique features per array. Depending on the demands of the experiment and the number of probes required per array, each wafer, can be cut into tens or hundreds of individual arrays.

Other methods may be used to produce the biochip. The biochip may be a Langmuir-Bodgett film, functionalized glass, germanium, silicon, PTFE, polystyrene, gallium arsenide, gold, silver, membrane, nylon, PVP, or any other material known in the art that is capable of having functional groups such as amino, carboxyl, Diels-Alder reactants, thiol or hydroxyl incorporated on its surface. These groups may then be covalently attached to crosslinking agents, so that the subsequent attachment of the nucleic acid ligands and their interaction with target molecules will occur in solution without hindrance from the biochip. Typical crosslinking groups include ethylene glycol oligomer, diamines, and amino acids.

The inventive aptamer biochip of the present invention differs from the prior art chips described above, in that it is manufactured by an enzymatic process that generates aptamers on the chip in situ through enzymatic synthesis, followed by self-addressed immobilization. It has been found that by manufacturing a biochip in which each feature comprises a pair of probes, the aptamers can be generated on the chip in situ through enzymatic synthesis.

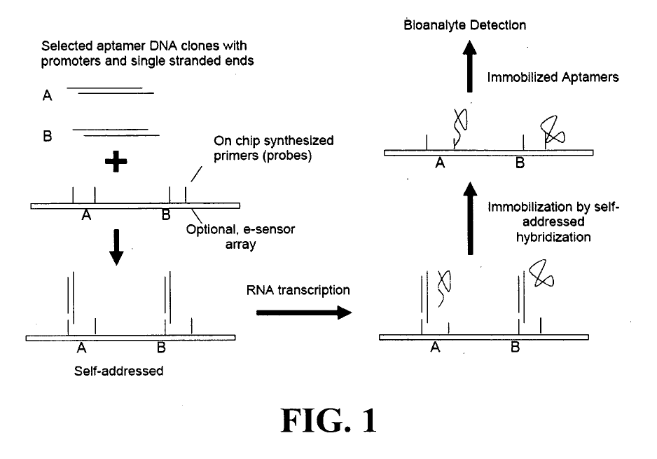

As illustrated in FIG. 1, the present biochip has two probes at each feature. FIG. 1 shows an example of on-chip synthesis of aptamer-addressable arrays for bioanalyte detection. In the illustration, two aptamer species, A and B, are depicted. Illustratively, step 1 is the hybridization of aptamer DNA clones onto chip. Step 2 uses RNA polymerase (transcription) to produce RNA aptamers in situ locally. Step 3 is the immobilization of RNA aptamers on chip and optional removal of aptamer DNAs. Finally, step 4 shows on-chip optical or electrical detection.

In greater details, FIG. 1 shows a first probe for the incoming aptamer dsDNA template. The second probe is for RNA aptamers to be synthesized in situ. Preparation of this novel biochip involves the creation of a probe chip which is manufactured according to known methods, such as those already described herein. Once the probe biochip has been created, the aptamer chip is prepared by contacting the probe chip with aptamer dsDNA clones that are selected with promoters, and single stranded ends of unique sequences are hybridized to one set of the probes on the probe biochip.

RNA polymerase, primers, and NTPs in buffer solution are then applied to the biochip, initiating RNA transcription and generating RNA aptamers according to the instruction of aptamer dsDNA clones. The in situ generated RNA aptamers may be immobilized by self-addressed hybridization to another set of adjacent probes. The resulting product is an aptamer biochip. Further, the dsDNA can be removed by applying DNase, leaving only RNA aptamers on chip.

The manufacturing procedure of an aptamer biochip involves the following steps. First, aptamer dsDNA clones (library) with single stranded ends of unique sequences are prepared. From this, the DNA probe chips may be generated. Then, dsDNA may be hybridized to the probes on the chip. RNA'aptamers are then generated by RNA polymerase (i.e., RNA transcription). The RNA aptamers may then be immobilized by self-addressed hybridization; and then optionally removing the dsDNA, thereby generating an aptamer chip.

The aptamer biochip may then be used for bioanalyte detection. For example, a blood sample may be contacted to the aptamer biochip. On-chip bioanalyte detection may then be conducted using known processes which may include electric signal (capacitance/conductance/CHEM-FET); fluorescence, phosphorescence, evanescence; interferometry; surface plasmon resonance (SPR); SERS; or other known methods. A CHEM-FET sensor refers to chemically sensitive field effect transistor that uses different gate materials to sense chemical species such as analyte vapors.

With reference to FIG. 2, addressable aptamers are generated in situ as shown in FIG. 1 on a biochip equipped with electrodes. After applying sample, the presence of target bioanalytes is detected on chip by changes in capacitance and/or conductance upon their binding to aptamers on chip. The electric data are then digitized and analyzed by computer.

When using SERS analysis for analyte detection, molecules located near metal are excited by the surface plasmon generated by interaction between the excitation light and the metallic surface. Specifically, it has been observed that molecules near roughened silver surfaces show enhanced Raman scattering of as much as six to seven orders of magnitude. The SERS effect is related to the phenomenon of plasmon resonance, wherein a metal surface exhibits a pronounced optical resonance in response to incident electromagnetic radiation, due to the collective coupling of conduction electrons in the metal. In essence, metal surface can function as miniature “dish-antenna” to enhance the localized effects of electromagnetic radiation. Molecules located in the vicinity of such surfaces exhibit a much greater sensitivity for Raman spectroscopic analysis. In ideal condition, the surface plasmon has several orders of magnitude higher intensity of electromagnetic field compared to the intensity of electromagnetic field of excitation light, and hence the Raman scattering by the molecules are several orders stronger than what the excitation light would have generated without the surface enhancements.

SERS techniques can give strong intensity enhancements by a factor of up to 10¹⁴ to 10¹⁶ or 10¹⁸, preferably for certain molecules (for example, dye molecules, adenine, hemoglobin, and tyrosine), which is near the range of single molecule detection. Generally, SERS is observed for molecules found close to silver or gold nanoparticles (although other metals may be used, but with a reduction in enhancement). The mechanism by which the enhancement of the Raman signal is provided is from a local electromagnetic field enhancement provided by an optically active nanoparticle. Current understanding suggests that the enhanced optical activity results from the excitation of local surface plasmon modes that are excited by focusing laser light onto the nanoparticle. SERS gives all the information usually found in Raman spectra; it is a sensitive vibrational spectroscopy that gives structural information on the molecule and its local interactions.

In another embodiment, protein target molecules bound to nucleic acid ligands on the surface of the biochip will be detected by the addition of chemicals that non-specifically bind to all proteins but not to nucleic acids. More generally, such agents bind to proteins preferably over nucleic acids. Any fluorescent chemical that is known in the art to bind proteins non-specifically will be suitable. Suitable examples include the dyes Nanorange and Cytoprobe, available from Molecular Probes, Inc.

In another embodiment, specific detection of protein that is covalently (or noncovalently) coupled to immobilize aptamer can be achieved by taking advantage of the different reactivities of nucleic acid and protein functional groups. Nucleic acids have no strong nucleophiles, whereas lysine and cysteine side-chains provide active nucleophiles to proteins. Lysine is a moderately abundant amino acid, comprising 4-6% of the side chains of most proteins. Cysteine varies considerably more in its abundance, and is often sequestered in disulfide bonds with other cysteine residues, rendering it less available for reaction.

Accordingly, the chemistry of protein modification through lysine residues is well-developed. A large number of fluorophores or other tagging agents have been developed which react with lysine. The most common chemistries rely on the reaction of lysine with hydroxysuccinamide (NHS) esters, isothiocyanates, or in a variety of aldehyde reactions.

In another embodiment, target molecules bound to nucleic acid ligands will be detected on the biochip surface through the use of a sandwich assay. This method is well known to those skilled in the art. A sandwich assay uses antibodies that recognize specific bound target molecules, preferably binding at a site distinct from that recognized by the nucleic acid ligand. In such sandwich assays, the antibodies may be fluorescently labeled, or the bound antibodies may themselves be detected by contacting the biochip with fluorescently labeled protein A, which binds all immunoglobulins. Alternatively, secondary antibodies specific for the immunoglobulin subtype of the first (primary) antibody will be contacted with the biochip. The secondary antibodies may be fluorescently labeled, or they may be conjugated to a reporter enzyme, which enzyme catalyses the production of a detectable compound. Sandwich assays have the potential to greatly amplify the detectable signal, in this case by the ability of the secondary antibody to bind to multiple sites on the primary antibody. All variations of the sandwich assay known in the art are contemplated in the present invention.

In a related sandwich assay embodiment, the bound target molecule will be detected by the use of a second nucleic acid ligand, which binds to a site on the bound target distinct from that recognized by the biochip-bound nucleic acid ligand. As described in the paragraph above, the second nucleic acid ligand may be fluorescently labeled, or it may be conjugated to biotin, allowing fluorescently labeled avidin, or an avidin conjugated reporter enzyme, to then bind to the bound second nucleic acid ligand. Alternatively, the first and second nucleic acid ligands may be labeled in an appropriate manner so that they form an energy transfer pair.

In another embodiment, target molecule binding will be detected using a competition assay, well known to those skilled in the art. Following contacting of the biochip-bound nucleic acid ligands, i.e., aptamers, with the test mixture, a solution containing a predetermined amount of each target for which binding data is sought is added. These target molecules are fluorescently labeled by any of the ways known in the art in order to permit their detection. The labeled target molecules compete for binding to the immobilized nucleic acid ligand. Equilibrium will be established, and the amount of labeled molecule bound at each site will be used to calculate the amount of each target molecule contained within the original test mixture.

In another embodiment, protein enzymes bound to aptamers can be detected by an assay of enzyme activity.

Another detection method for use with the present invention includes chemical field effect transistor (CHEM-FET) technology. This technology uses the local change in chemical potential that is created upon the binding of target molecule to its ligand. In this technology, an insulative silica “gate” is placed between two n-type semiconductors, forming a biochip. Current will flow from one semiconductor to the other when a conducting channel is formed in the gate and a potential difference is applied. Such channels could be opened when an ionic species binds to the silica gate. In another method, ligands are bound to discrete regions of one of the semiconductors via photoactivation of derivatizing groups. The biochip is then contacted with a mixture containing target molecules. Binding of a target molecule to a ligand leads to a net loss or gain of ions at that location of the biochip. The ions locally alter the conductance at this location, which in turn leads to a change in the drain current in this area of the biochip. If the biochip is configured in such a way that current drains will be measured in discrete locations on the biochip (multigated CHEM-FET), then spatial and quantitative assessment of target binding will be achieved. Advances in the art should permit the scaling up of this technology to independently and accurately measure thousands of spatially discrete changes in drain current.

Another bioelectric change that can be measured using CHEM-FET is the photoinduced electron transfer which occurs in double-stranded DNA. The degree of double-strandedness of each nucleic acid ligand may change when a target molecule is bound. Changes in the extent of double-helicity will lead to localized changes in drain currents in a CHEM-FET biochip that is being illuminated. If the CHEM-FET biochip is read before and after contact with the target mixture, then detecting these differences will reveal the sites and extent of target molecule binding.

In another embodiment, target molecule binding will be detected through surface plasmon resonance (SPR). In this technique, nucleic acid ligand is immobilized on a gold or silver film on the surface of a prism; the metal film is then incubated in the appropriate liquid medium. Therefore, the metal film is at the prism-liquid interface. Light is directed through the prism towards the medium, and above a critical angle, total internal reflection of the light occurs. Above this critical angle, an evanescent wave extends into the medium by a distance that is approximately equal to the wavelength of the incident light. The evanescent wave excites free oscillating electrons, termed surface plasmons, in the metal film, and causes them to resonate. Energy is absorbed from the evanescent wave by the electrons during this process, thereby reducing the intensity of the internally reflected light. The angle at which total internal reflection, and hence resonance, occurs is sensitive to changes in the refractive index of the medium immediately adjacent to the metal film. When a target molecule binds to a nucleic acid ligand on the surface of the film, the refractive index at this site changes, and the angle needed to cause resonance changes also. Thus in order to detect target molecule binding, a detector system is arranged in which the angle of incident light is varied, and the intensity of the reflected light is measured. Resonance occurs when the intensity of the reflected light is at a minimum. Measuring the change in angle of incident light needed to bring about resonance at specific sites on the film in the presence of a test mixture can then yield information about where a binding reaction has occurred on the surface of the film. A device for measuring SPR called BIAcore7 is commercially available from Pharmacia Biosensors.

In another embodiment, the formation of nucleic acid ligand-target complex will be detected by mass spectroscopy. The surface of the biochip will be irradiated in a spatially restricted and sequential way using a laser that is capable of ionizing the biological material on the biochip. The mass of the ionization products will be detected by mass spectroscopy, and comparison with the mass of ionization products of the same unbound ligands will reveal where target is bound. This technique is known in the art as Matrix Absorption/Laser Desorption and Ionization (MALDI) Time of Flight Mass Spectroscopy. The nucleic acid ligands and the targets in this embodiment can be covalently associated through the use of photoactive crosslinking groups on the nucleic acid ligand, as described above.

Detection may also be done via Atomic Force Microscopy (AFM) and Scanning-Tunneling Microscopy (STM). These related methods are well known in the art as techniques useful for describing the topology of surfaces at the nanometer level. Hence, advances in these techniques will make them suitable for detecting sites on a biochip where target molecule has been bound by a nucleic acid ligand. Atomic force microscopy (AFM) uses a non-metallic probe which is scanned over the surface of interest, in this case a biochip. The probe is moved close to the surface so that the probe is subject to electron-repulsive interactions with the material bound to the surface. Repulsion leads to the deflection of a cantilever upon which the probe is mounted, and this deflection is measured by a laser-photodiode detection system. The surface under examination is mounted on a stage, which stage is coupled to the deflection detection system by a computer. When the probe is deflected, the stage is lowered, allowing the probe to trace out a “contour map” of electron density for the surface. Using this technique, a reference map for a nucleic acid biochip in a buffer will be prepared. This will be compared with a map obtained from a nucleic acid biochip that has been incubated with a test mixture. Comparison of the two maps will allow detection of sites on the biochip where target molecule has bound.

Scanning tunneling microscopy (STM) uses a metallic probe which is scanned over a surface of interest. When the probe approaches the material bound to the surface, electrons can “tunnel” between the probe and the material, and the resulting current can be detected. The probe is scanned over the surface, and the vertical position of the probe is constantly varied to permit tunneling. This, as in AFM, gives a map of electron density, which map will be used as described in the above paragraph to detect target molecule binding on a nucleic acid ligand biochip.

In another embodiment, target molecule binding will be assessed by monitoring changes in the degree of double-strandedness of each nucleic acid ligand. It is known that nucleic acid ligands undergo structural changes upon binding to target, such as the formation, or expansion, of double stranded regions. In the instant invention, these changes will be detected by adding a fluorescent intercalating dye, such as ethidium bromide, to the biochip, and measuring fluorescence levels at each location on the biochip in the presence and absence of the test mixture.

In another embodiment, nucleic acid ligands containing a constant sequence associated with the binding site for the target molecule may be localized to specific regions of a biochip. The biochip-bound nucleic acid ligands may then be hybridized with an oligonucleotide complementary in sequence to the constant region. Contacting this biochip with a test mixture can lead to displacement of oligonucleotide from nucleic acid ligands that bind to their target molecule.

Further, a biochip may be synthesized upon which the complementary oligonucleotide is immobilized. The nucleic acid ligands can then be deposited at specific locations on the biochip, whereupon they will become associated with the oligonucleotide by base pairing. The biochip can then be contacted with the test mixture. Target molecule binding will lead to the disruption of base pairing between the nucleic acid ligand and the support bound oligonucleotide, and hence displacement of the nucleic acid ligand from the biochip can occur.

In the preceding embodiments, the displaced nucleic acid may be labeled with, for example, fluorescein, tetramethylrhodamine. Texas Red, or any other fluorescent molecule known in the art, leading to a decrease in fluorescence intensity at the site of target molecule binding. The level of label detected at each address on the biochip will then vary inversely with the amount of target molecule in the mixture being assayed. Alternatively, the nucleic acid ligand and the oligonucleotide constitute an energy transfer pair. For example, one member of the pair will be labeled with tetramethylrhodamine and the other will be fluorescein labeled. The fluorescein-based fluorescence of such a complex is quenched when illuminated with blue light, as the green light emitted by the fluorescein will be absorbed by the tetramethylrhodamine group; the rhodamine-based fluorescence of this complex is not quenched. Separation of the two halves of this energy transfer pair occurs upon target molecule binding, and leads to a change in the emission profile at such sites on the biochip. The displacement of the tetramethylrhodamine labeled molecule will lead to the sudden appearance of fluorescein-based fluorescence at this site on the biochip, with the concomitant loss of rhodamine-based fluorescence. The simultaneous change in two different emission profiles will enable ratiometric imaging of each site to be performed, allowing sensitive measurement of target molecule binding. It is clear to those skilled in the art that any energy transfer pair can be used in this embodiment, providing that they have appropriately matched excitation and emission spectra.

In an alternative embodiment, the displaced nucleic acid is conjugated to one member of an affinity pair, such as biotin. A detectable molecule is then conjugated to the other member of the affinity pair, for example avidin. After the test mixture is applied to the biochip, the conjugated detectable molecule is added. The amount of detectable molecule at each site on the biochip will vary inversely with the amount of target molecule present in the test mixture. In another embodiment, the displaced nucleic acid will be biotin labeled, and can be detected by addition of fluorescently labeled avidin; the avidin itself will then be linked to another fluorescently labeled, biotin-conjugated compound. The biotin group on the displaced oligonucleotide can also be used to bind an avidin-linked reporter enzyme; the enzyme will then catalyze a reaction leading to the deposition of a detectable compound. Alternatively, the reporter enzyme will catalyze the production of an insoluble product that will locally quench the fluorescence of an intrinsically-fluorescent biochip. In another embodiment of the displacement assay, the displaced oligonucleotide will be labeled with an immunologically-detectable probe, such as digoxigenin. The displaced oligonucleotide will then be bound by a first set of antibodies that specifically recognize the probe. These first antibodies will then be recognized and bound by a second set of antibodies that are fluorescently labeled or conjugated to a reporter enzyme. Many variations on these examples are well known to those skilled in the art.

In variations of the preceding embodiments, the nucleic acid ligand will not contain a constant sequence region as described above. In these embodiments, the oligonucleotide will have a sequence that is complementary to all, or part of, the nucleic acid ligand. Thus, each nucleic acid ligand will bind an oligonucleotide with a unique sequence. The oligonucleotides can be displaced from biochip-localized nucleic acid ligands as described above upon target binding. Alternatively, the oligonucleotides will be localized to specific locations on the biochip as described above, which will in turn result in the specific localization of nucleic acid ligands by complementary base-pairing to the oligonucleotides. Target molecule binding will displace the nucleic acid ligand from the biochip in this case, as described above. In each case, the oligonucleotide and/or the nucleic acid ligand can be labeled as described above.

In other embodiments, nucleic acid ligands may be localized to specific regions of the biochip. Following contacting with the test mixture, the biochip can then be contacted with a solution containing either (i) oligonucleotide with sequence complementary to the constant region of the nucleic acid ligand; or (ii) oligonucleotides with sequence complementary to all or part of each nucleic acid ligand, which ligand does not contain a constant sequence region as described above in this section. In these cases, binding of target will prevent the subsequent binding of oligonucleotide. Again, the oligonucleotide and the nucleic acid ligand can be labeled as described above in this section to monitor the binding of oligonucleotide.

Current technologies of aptamer biochips immobilize aptamers themselves directly onto chip surface by various means. There are limitations associated with these technologies. For example, “ink-jet” spotting-based array production is limited by the size of the spots. The on-chip synthesis of addressable aptamer arrays alleviates these problems because much higher densities of aptamers can be made using pre-fabricated high density DNA arrays.

Further, other advantages include the synthesis of long molecules on the array: Current high density array method can only generate nucleic acid of <50 nt, the present method allows for the generation of molecules of greater than 100 nt or even 1000 nt. Because the aptamers are self-addressed in a single reaction chamber, the fabrication process is much more simple and cost effective, as no individual aptamer sample preparation and immobilization is required. As well, antibodies are widely used as affinity capturing agents. However, they are produced from animal products, making this process is very time consuming and costly.

Generally, it is not feasible to generate an antibody. Aptamers, on the other hand, are produced in vitro, and therefore provide numerous advantages including easy production and easy chemical modification, thereby making the present process more efficient and less expensive than prior art methods.

### Example

### Preparation of Probe Array

A DNA array is used as a substrate to generate an aptamer array. A DNA array can be built according to known skills in the art, such as spot array and in situ synthesized high density DNA array. For example, photolithography technologies using a photomask and photoactive protecting groups could also be used.

To build an aptamer array, two types of probe sequences are needed for each aptamer sequence. One probe is used to capture the incoming dsDNA clones (the aptamer template), and the other is to capture RNA aptamers to be synthesized by RNA transcription from the adjacent DNA template. The exemplary design for one feature on the surface of this probe array is depicted in FIG. 1a below. In this design, one standard photolithography feature set with dimension of 50 μm×50 μm, for example, contains 9 smaller features, 4 of them contain the same probe molecules for the DNA template and 5 of them contain the same probe sequence to capture the RNA aptamer molecules. The capture probe sequences are unique and have designed to minimize cross hybridization. Accordingly, the photomask is designed to accomplish this photolithographic synthesis.

Selection of Aptamer dsDNA Clones by SELEX

Standard SELEX procedure that is known in the art is used to generate dsDNA clones with minor modification of the template sequences. Unique single stranded ends will be added next to the T7 promoter sequence for hybridization of dsDNA clones to the capture probes that are synthesized on the chip surface. These clones are selected against various biological targets of interest including biomarkers for cardiovascular diseases, cancers, or infectious diseases.

Note that T7 RNA polymerase is very specific for T7 promoters and it does not recognize DNA from other sources, since these promoter sequences are very rare. Also, termination signals for T7 RNA polymerase are rare too, so long transcripts can be made without premature truncation. The T7 RNA polymerase is about 5 times faster that E. coli RNA polymerase, so genes controlled by T7 promoters can be overexpressed. Also, note that T7 lysozyme is an inhibitor of T7 RNA polymerase.

Immobilization of Selected Aptamer dsDNA Clones to Array Surface.

The aptamer dsDNA clones selected against various biological targets of interest by for the SELEX procedure described above are introduced onto the surface of the probe array in proper buffer. For example, a mixture of many dsDNA clones are hybridized on the chip (containing on-chip synthesized complementary probes) at 40° C. in PBST (2× phosphate buffer saline (PBS), 0.05% Tween-20) buffer for 1 h. The chip was then washed 3 times with 0.5×PBS prepared with DEPC-treated water (DEPC=diethylpyrocarbonate) to remove excess reagents and non-bound dsDNA clones. This will also clean the surface to make sure it is nuclease-free for the next transcription step. Other molecular hybridization conditions know in the art can alai be used.

RNA Transcription and Immobilization of RNA Aptamers

This step of RNA transcription can be accomplished by adapting various procedures reported in the literature or procedures for commercially available in vitro transcription kit. For example, Ambion MEGAscript® can be used.

Prepare Reagents

Place the RNA Polymerase Enzyme Mix on ice; warm 4 nucleotide solutions (ATP, CTP, GTP, and UTP) quickly in your hands (wearing gloves) until they are completely in solution, keep on ice; to covalently immobilize RNA aptamers on the chip surface, modified NTP such as 5-bromo UTP can be added in the RNA transcription. Vortex 10× reaction buffer until it is completely thawed, keep it at room temperature.

Mix Reagent and Add the Reagents onto Surface.

In a tube, combine and mix the following to assemble the transcription reaction: DEPC-H₂O (14 μL), 10× buffer (4 μL), ATP (4 μL), GTP (4 μL), CTP (4 μL), UTP (4 μL), and T7 polymerase (4 μL). Mix well. Apply the reagent mix to the chip surface where DNA clones (templates) are specifically captured.

Incubation and Aptamer Immobilization

The biochip was incubated at 37° C. for 2 hours in a humidified chamber. The RNA aptamers synthesized are captured by adjacent capture probe molecules and thus immobilized through self-addressed hybridization.

Wash

At the end of incubation, the chip is washed with buffer (0.1×PBS) to remove excess reagents and unbound RNA aptamers. Preserve the aptamer chip in 0.1×PBS containing RNase inhibitor available from Promega or other vendors.

Crosslinking of RNA Aptamers to Probes (Optional)

To covalently immobilize RNA aptamers on the chip surface, modified NTP such as 5-bromo UTP can be added in the RNA transcription. After hybridization to capture probe on surface, ultraviolet light (308 nm) is applied to crosslink RNA aptamer to the probe via photochemistry of bromouridine.

Removal of dsDNA (Optional)

There are numerous commercially available kits for removal of dsDNA using DNase. The following procedure is adapted from Promega RQ1 RNase-Free DNase kit.

Reagents

10× Reaction Buffer (M198A): The RQ1 DNase 10× Reaction Buffer provided with this enzyme has a composition of 400 mM Tris-HCl (pH 8.0), 100 mM MgSO4 and 10 mM CaCl2.

Enzyme Storage Buffer: RQ1 DNase is supplied in 10 mM HEPES (pH 7.5), 50% glycerol (v/v), 10 mM CaCl₂ and 10 mM MgCl₂.

Storage Temperature: Store at −20° C. Avoid exposure to frequent temperature changes. See the expiration date on the Product Information Label.

Stop Solution (M199A): 20 mM EGTA (pH 8.0).

Procedure

Set up the DNase digestion reaction as follows: (i) Water or TE buffer 10-80 μl; RQ1 RNase-Free DNase 10× Reaction Buffer 10 μl; RQ1 RNase-Free DNase 1 u/μg; and nuclease-free water to a final volume of 100 μl; (ii) incubate at 37° C. for 30 minutes; (iii) add 1 μl of RQ1 DNase stop solution to terminate the reaction; and (iv) wash the biochip with buffer to remove excess reagents.

Applications of Aptamer Biochips

Aptamers have been touted to be a new generation of affinity agents with functional characteristics that are similar to antibodies. For example, aptamers can bind to their cognate targets with nanomolar or picomolar binding affinity, matching or even superseding the affinities of antibodies. For these inherent properties, aptamers hold great promise in various biological and biomedical applications. For instance, aptamers can serve as designer matrices for affinity chromatography, biosensors, therapeutic agents, genetic switches, allosteric ribozymes, capture agents for microarray biochips in molecular detection, and so on. That aptamers serves as capture agents on the surface of a microarray biochips is most relevant to this invention and all of the potential applications associated with protein microarray including antibody microarray and antigen microarray are applicable to aptamer biochips in this invention. Therefore, potential applications of the aptamer biochip generated from this invention include protein profiling (proteomics), drug screening (drug discovery), biomarker discovery and analysis (diagnostics), biosensing (food analysis, environmental analysis and biosafety), and basic research.

Proteomics: Protein Profiling and Microarray

With finish of the Human Genome Project and dawn of proteomics era, new tools are needed to analyze biological functions of proteins expressed in any given organism. Proteomics as a new scientific field is facing a lot of challenges. For example, numerous biological transformations have been associated with proteins after translation affecting the functions of proteins. These include posttranslational modifications, such as phosphorylation, splicing, etc. In addition, the dynamic range of protein expression in a given organism is very broad. More sensitive detection and analysis technologies are needed to address the dynamic range problem. Thus, to profile proteins in a given organism is quite challenging. Aptamer biochip will alleviate this burden by providing potentially more sensitive detection technology, high density array, and high volume of information.

Drug Discovery and Target Validation

Aptamers can be designed to bind target proteins specifically and inactivate their targets. This property predestines aptamers as surrogate ligands in high throughput screening (HTS) for drug discovery. The structural and functional information stored in the aptamer can be converted into a small molecule drug that can inhibit the target protein function in a similar fashion to aptamer and validate the drug target. Therefore, the aptamer biochip in this invention will be a valuable tool for drug discovery and target validation.

Diagnostics: Biomarker Discovery and Protein Signature Analysis

Biomarker discovery for diseases pose big challenge to diagnostics research and development. Aptamer biochip provides a tool to discover new markers for diseases such as cancers and heart diseases. Specially, aptamer biochip offers pattern analysis of protein markers involved in a disease stage, providing a better overall picture for diagnostic purpose. Thus, biomarker signature analysis is becoming a trend in diagnostics industry.

Biosensening

The high binding affinity of aptamers to their cognate targets can be used in biosensing as well. For example, aptamer beacons can be built onto chip. Upon binding to their targets, the binding events will trigger fluorescent emission of specific wavelength. This can be used in food and environmental analysis. It is also useful for remote biosensing in a bio-threatening environment. Thus, the aptamer biochip can have potential uses in biosensing.

Basic Research

Basic research using aptamers as detecting agents in affinity assay such as ELISA, microarray, and immunoprecipitation, can be used to identify previously unknown protein on cell surface, or inside cells, especially these new disease markers. They can also be used detect proteins with very low abundance. Once these new proteins are identified their biological functions will be studied. Therefore, aptamer biochips are useful tools for basic research.

This application may disclose numerical range limitations that support any range within the disclosed numerical ranges even though a precise range limitation is not stated verbatim in the specification because the embodiments of the invention could be practiced throughout the disclosed numerical ranges. Finally, the entire disclosure of the patents and publications referred in this application, if any, are hereby incorporated herein in entirety by reference.

1. A method for preparing an aptamer biochip, comprising:
obtaining a double stranded DNA clone comprising a single stranded end having a unique sequence;
obtaining a probe chip having an array of features, wherein a feature comprises at least two kinds of probes, the at least two kinds of probes comprising a first probe and a second probe;
hybridizing the single stranded end of the double stranded DNA clone to the first probe;
generating an RNA aptamer by transcription of the double stranded DNA clone hybridized to the first probe;
immobilizing the RNA aptamer on the second probe; and
forming the aptamer biochip
wherein the first probe is configured to capture the single stranded end of the double stranded DNA clone and the second probe is configured to capture the RNA aptamer.

2. The method of claim 1, wherein the double stranded DNA clone further comprises a promoter.

3. (canceled)

4. The method of claim 1, wherein the RNA aptamer attaches to the second probe simultaneously during transcription.

5. The method of claim 1, wherein the double stranded DNA clone comprises a library of double stranded DNA clones, wherein at least a portion of the double stranded DNA clones has a common promoter sequence and a unique tag sequence.

6. The method of claim 1, further comprising removing unbound DNA and/or RNA from the aptamer biochip.

7. The method of claim 1, further comprising removing unbound double stranded DNA clone from the aptamer biochip.

8. The method of claim 3, wherein transcription is initiated by applying an RNA polymerase to the chip.

9. The method of claim 1, wherein the generating the RNA aptamer is done in situ on the probe chip by RNA transcription such that the RNA aptamer generated is immobilized on the second probe simultaneously during the RNA transcription.

10-53. (canceled)

54. The method of claim 1, wherein obtaining the double stranded DNA clone comprises generating the double stranded DNA clone with systematic evolution of ligands by exponential enrichment (SELEX).

55. The method of claim 1, further comprising coupling a protein to the immobilized RNA aptamer.

56. The method of claim 55, further comprising binding a fluorophore to the protein.

57. The method of claim 1, further comprising forming a sandwich assay with the aptamer.

58. The method of claim 57, further comprising binding a fluorophore to the sandwich assay.

59. The method of claim 57, further comprising binding a second nucleic acid ligand to the sandwich assay.

60. The method of claim 59, wherein the second nucleic acid is conjugated with biotin.

61. The method of claim 1, wherein the generating of the RNA aptamer and the immobilizing the RNA aptamer on the second probe includes in-situ synthesis of the RNA aptamer on the first probe.
